(19) United States
(12) Patent Application Publication
(10) Pub. No.: US 2026/0096778 A1
(43) Pub. Date: Apr. 9, 2026

(54) Conduction-Based Contact Detection for Physiological Sensors

(71) Applicant: iRhythm Technologies, Inc., San Francisco, CA (US)

(72) Inventor: Steven Robert Keyes, San Francisco, CA (US)

(73) Assignee: iRhythm Technologies, Inc., San Francisco, CA (US)

(21) Appl. No.: 19/270,133

(22) Filed: Jul. 15, 2025

Related U.S. Application Data
(60) Provisional application No. 63/703,444, filed on Oct. 4, 2024.

(51) Int. Cl. A61B 5/00; A61B 5/0531; A61B 5/1455; A61B 5/28

(52) U.S. Cl. CPC A61B 5/6844; A61B 5/0531; A61B 5/14552; A61B 5/28

(57) ABSTRACT

Techniques for conduction-based contact detection for physiological sensors are described. In an example, a wearable device includes a contact-based sensor configured to collect physiological data of a user and at least one electrode positioned proximate to the contact-based sensor, such that a contact status of the electrode corresponds to a contact status of the sensor. The wearable device includes a contact detection circuit that is configured to determine a contact condition of the contact-based sensor that indicates whether the sensor is in sufficient contact with the skin surface based on an electrical characteristic of a conduction path between the at least one electrode and a skin surface of the user. In this way, the techniques described herein support reliable detection of sensor contact quality through conduction-based measurements, which enables wearable devices to ensure accurate physiological data collection while optimizing power consumption and improving an overall user experience.

### RELATED APPLICATIONS

This application claims priority to U.S. Provisional Application No. 63/703,444, titled Conducted Leads-Off for Photoplethysmography Poor Contact Detection, filed Oct. 4, 2024, which is hereby incorporated by reference in its entirety.

### BACKGROUND

Wearable devices often include integrated sensors to monitor physiological parameters of a user to provide various insights. For instance, wearable devices include non-invasive sensors that rely on consistent contact with a skin surface of a user to collect and analyze vital signs, activity levels, and other health-related metrics in real-time as users go about daily activities. However, poor or inconsistent contact of these sensors with the skin surface can lead to inaccurate readings, data gaps, and unreliable health insights that may result in incorrect clinical decisions or missed health events. These contact-related issues can significantly offset the convenience and continuous monitoring advantages provided by wearable devices and reduce utility as reliable health monitoring tools in everyday settings.

### BRIEF DESCRIPTION OF THE DRAWINGS

FIG. 1 is a block diagram of a non-limiting example of an environment that is operable to employ techniques for conduction-based contact detection for physiological sensors as described herein.

FIG. 2 depicts a non-limiting example of a monitoring device.

FIG. 3 depicts a non-limiting system in an example implementation of conduction-based contact detection for physiological sensors showing operation of the contact detection system of FIG. 1 in more detail.

FIGS. 4a, 4b, and 4c depict nonlimiting examples of conduction-based contact detection for physiological sensors in which various contact conditions are detected by a wearable device.

FIG. 5 depicts a nonlimiting example of conduction-based contact detection for physiological sensors in which an electrode arrangement supports determination of multiple electrical conditions.

FIG. 6 depicts a nonlimiting example of conduction-based contact detection for physiological sensors in which a user interface for a loss of contact scenario is shown.

FIG. 7 depicts a nonlimiting example of conduction-based contact detection for physiological sensors in which a user interface for a partial contact scenario is shown.

FIG. 8 depicts a flow diagram depicting an algorithm as a step-by-step procedure in an example implementation, one or more steps of which are performable by a processing device to determine and respond to a contact condition of a contact-based sensor.

FIG. 9 illustrates a flow diagram depicting an algorithm as a step-by-step procedure in an example implementation, one or more steps of which are performable by a processing device to detect contact conditions of a wearable device with a skin surface.

### DETAILED DESCRIPTION

Contact-based sensors, such as those implemented by wearable devices, can collect data over extended observation periods to provide a variety of real-time insights into various health-related metrics. However, these sensors may frequently lose contact with a skin surface of a user, such as because of user movement, failed adhesives, improper device placement, perspiration, or environmental factors that can compromise integrity of a sensor-to-skin interface. These contact related issues can lead to inaccurate readings, data gaps, and unreliable health insights which offsets the benefits provided by these techniques. Accordingly, it is desirable to accurately determine when a contact-based sensor loses contact with a skin surface.

However, conventional techniques to detect sensor contact are limited. Some conventional approaches, for instance, analyze data collected by the sensor itself to attempt to identify instances of sensor dislodgment however these approaches are inaccurate and subject to error. For example, conventional techniques for detecting sensor contact for an optical sensor (e.g., a photoplethysmography (“PPG”) sensor) rely on optical signal analysis, which is susceptible to interference from factors such as ambient light, skin pigmentation, or foreign objects that may block the optical sensor while still allowing light reflection.

These limitations result in “false positives” where lapses in sensor contact are not detected and therefore wearable devices continue to collect inaccurate data. As such, conventional contact detection approaches struggle to accurately differentiate between poor sensor contact and genuine physiological changes. These limitations may undermine the utility of wearable devices as reliable health monitoring tools in everyday settings and further may cause unnecessary expenditure of computational resources to operate a sensor that is not in contact with a skin surface of a user.

Accordingly, techniques, methods, and systems for conduction-based contact detection for physiological sensors are described that improve contact detection relative to conventional approaches. The techniques described herein, for instance, leverage conduction-based leads-off detection circuitry to reliably detect sensor contact issues. By positioning one or more biopotential electrodes proximate to a contact-based sensor, such as a PPG sensor, a contact status of the electrodes can be correlated to a contact status of the sensor, offering a robust approach to contact detection that is not susceptible to optical interference.

Consider an example in which a smartwatch is equipped with a PPG sensor to collect heart rate and blood oxygen data of a user. The PPG sensor, for instance, includes an optical sensor that emits light into a skin surface of the user and measures an amount of light reflected or absorbed. The reflected or absorbed light varies with blood flow, and thus measurements collected by the optical sensor are usable to derive heart rate and blood oxygen levels. However, using conventional techniques, the smartwatch is unable to accurately detect when the PPG sensor loses contact with the skin, such as when a foreign object such as a shirt sleeve interferes with the optical signal. This results in inaccurate and/or missing PPG data which causes incorrect health insights.

To overcome these limitations, a wearable device incorporates a contact-based sensor, such as a PPG sensor, configured to collect physiological data of a user. The device further includes an electrode arrangement that includes at least one electrode, e.g., a biopotential electrode, positioned proximate to the contact-based sensor such that contact of the proximate electrode with the skin surface correlates to contact of the PPG sensor with the skin surface. For instance, when the proximate electrode maintains contact with the skin, this indicates that the PPG sensor is also in contact with the skin surface. Accordingly, the wearable device can leverage a contact status of the electrode to determine a contact quality of the PPG sensor.

The wearable device further includes a contact detection circuit coupled to the proximate electrode. The contact detection circuit, for instance, is configured to determine a contact condition of the contact-based sensor based on an electrical characteristic of a conduction path between the at least one electrode and the skin surface. In one or more examples, the contact condition indicates whether the contact-based sensor is in sufficient contact with the skin surface to collect accurate physiological data. Additionally or alternatively, the contact condition indicates a degree or quality of contact between the sensor and the skin and/or a likelihood that the sensor is able to collect accurate physiological data.

In an example to do so, the contact detection circuit injects a test current through one or more electrodes of the electrode arrangement, e.g., a driven electrode, and measures a voltage required to drive the test current through the conduction path, such as to a passive electrode of the electrode arrangement. A relatively high voltage value, for instance, corresponds to a relatively high impedance in the conduction path (e.g., based on a relatively low conductivity of air present between the electrodes and the skin surface) and thus indicates poor contact of the sensor with the skin surface. A relatively low voltage value corresponds to a relatively low impedance in the conduction path (e.g., based on a relatively high conductivity of the skin surface and subdermal tissue of the user) and thus indicates sufficient sensor contact.

Accordingly, when the monitored voltage exceeds a predetermined threshold the contact detection circuit can determine that the sensor has poor or no contact with the skin surface. Additionally or alternatively, the contact detection circuit can generate a quantification of contact quality between the sensor and the skin surface, such as based on a correlation between the voltage value and contact quality. In this way, the contact detection circuit can determine “how well” the sensor contacts the skin surface.

The wearable device is further operable to perform a variety of functionality based on the detected contact condition. For instance, the wearable device can cause presentation of an indication of the contact condition, such as to output one or more visual notifications, audio alerts, or haptic feedback that indicate the contact status, provide potential reasons for the contact status, and/or guide the user to improve sensor placement. Additionally or alternatively, the wearable device may modify analysis of the sensor data, such as weighting, filtering, or flagging portions of the physiological data based on the detected contact condition. In some examples, the wearable device adjusts operation of the sensor based on the contact condition, such as to suspend data collection from the sensor to conserve power and other computational resources.

Continuing with the above example, the smartwatch includes one or more electrodes positioned adjacent to the PPG sensor. The contact detection circuit monitors electrical characteristics between these electrodes and the skin surface of the user. If the smartwatch becomes disengaged from the skin surface, such as the user removing the smartwatch or a foreign object interferes with the optical signal, the smartwatch can efficiently detect the loss of contact. This allows the smartwatch to flag the data as potentially unreliable in real time, prompt the user to adjust the smartwatch, and/or suspend data collection from the PPG sensor to conserve power.

In this way, the techniques described herein overcome the limitations of conventional approaches by supporting reliable detection and/or evaluation of sensor contact in wearable devices. This enhanced contact detection capability enables accurate and continuous physiological monitoring, reduces false readings and data gaps, conserves computational resources, and improves reliability of health insights derived from wearable technology in real-world settings. Further discussion of these and other examples and advantages are included in the following sections and shown using corresponding figures.

In some aspects, the techniques described herein relate to a wearable device including: a contact-based sensor configured to collect physiological data of a user; at least one electrode positioned proximate to the contact-based sensor; and a contact detection circuit configured to determine a contact condition of the contact-based sensor based on an electrical characteristic of a conduction path between the at least one electrode and a skin surface of the user.

In some aspects, the techniques described herein relate to a wearable device, wherein the at least one electrode is positioned relative to the contact-based sensor such that contact of the at least one electrode with the skin surface correlates with contact of the contact-based sensor with the skin surface.

In some aspects, the techniques described herein relate to a wearable device, wherein to determine the contact condition the contact detection circuit is configured to inject a test current through the at least one electrode and measure the electrical characteristic as a voltage used to drive the test current.

In some aspects, the techniques described herein relate to a wearable device, wherein the contact detection circuit determines that the contact condition indicates that the contact-based sensor does not contact the skin surface responsive to detection of the voltage exceeding a threshold.

In some aspects, the techniques described herein relate to a wearable device, wherein the contact detection circuit determines that the contact condition indicates that the contact-based sensor is in contact with the skin surface responsive to detection of the voltage as being below a threshold.

In some aspects, the techniques described herein relate to a wearable device, wherein the contact condition includes a quantification of an amount of contact of the contact-based sensor with the skin surface based on a correlation to a value of the electrical characteristic.

In some aspects, the techniques described herein relate to a wearable device, wherein the wearable device is configured to cause output of an indication of the contact condition, the indication including one or more of a visual notification, audio alert, or haptic feedback.

In some aspects, the techniques described herein relate to a wearable device, wherein the wearable device is configured to perform data analysis on the physiological data collected by the contact-based sensor, the data analysis including weighting, filtering, or flagging one or more portions of the physiological data based on the contact condition.

In some aspects, the techniques described herein relate to a wearable device, wherein the contact-based sensor is a photoplethysmography (PPG) sensor and the at least one electrode includes a first electrode positioned adjacent to a first side of the PPG sensor and a second electrode positioned adjacent to a second side of the PPG sensor.

In some aspects, the techniques described herein relate to a method implemented by a wearable device attachable to a skin surface of a user, the method including: injecting a test current through a first electrode positioned adjacent to a contact-based sensor, the contact-based sensor configured to collect physiological data via contact with the skin surface; determining an electrical characteristic that includes a voltage value to drive the test current to a second electrode through a conduction path between the first electrode, the skin surface, and the second electrode; and determining a contact condition of the contact-based sensor based on the voltage value that indicates whether the contact-based sensor is in contact with the skin surface.

In some aspects, the techniques described herein relate to a method, wherein the first electrode is positioned relative to the contact-based sensor such that contact of the first electrode with the skin surface correlates with contact of the contact-based sensor with the skin surface.

In some aspects, the techniques described herein relate to a method, wherein the contact condition further includes a quantification of an amount of contact of the contact-based sensor with the skin surface based on a correlation to the voltage value.

In some aspects, the techniques described herein relate to a method, further including causing the contact-based sensor to pause collection of the physiological data responsive to a determination that the contact-based sensor is not in contact with the skin surface.

In some aspects, the techniques described herein relate to a method, further including causing presentation of an indication of the contact condition, the indication including one or more visual notifications, audio alerts, or haptic feedback.

In some aspects, the techniques described herein relate to a method, wherein the indication further includes diagnostic information about a cause of the contact condition and user guidance to improve contact between the contact-based sensor and the skin surface.

In some aspects, the techniques described herein relate to a method, wherein the contact-based sensor is a photoplethysmography (PPG) sensor, and one or more of the first electrode or the second electrode are integrated into an electrocardiogram (ECG) measurement component.

In some aspects, the techniques described herein relate to a contact detection system including: a contact-based sensor configured to collect physiological data via contact with a skin surface; an electrode arrangement that includes at least one proximate electrode positioned proximate to the contact-based sensor such that contact of the at least one proximate electrode with the skin surface correlates with contact of the contact-based sensor with the skin surface; and one or more processors configured to determine a contact condition of the contact-based sensor based on an electrical characteristic of a conduction path between the at least one proximate electrode and the skin surface.

In some aspects, the techniques described herein relate to a contact detection system, wherein the electrode arrangement includes a drive electrode configured to drive a test current and a passive electrode configured to detect the electrical characteristic resulting from the test current.

In some aspects, the techniques described herein relate to a contact detection system, wherein the electrode arrangement includes three or more electrodes and the one or more processors are configured to identify whether each of the three or more electrodes maintain contact with the skin surface.

In some aspects, the techniques described herein relate to a contact detection system, wherein the contact-based sensor includes one or more of a photoplethysmography (PPG) sensor, a temperature sensor, a heat flux sensor, or an optical sensor.

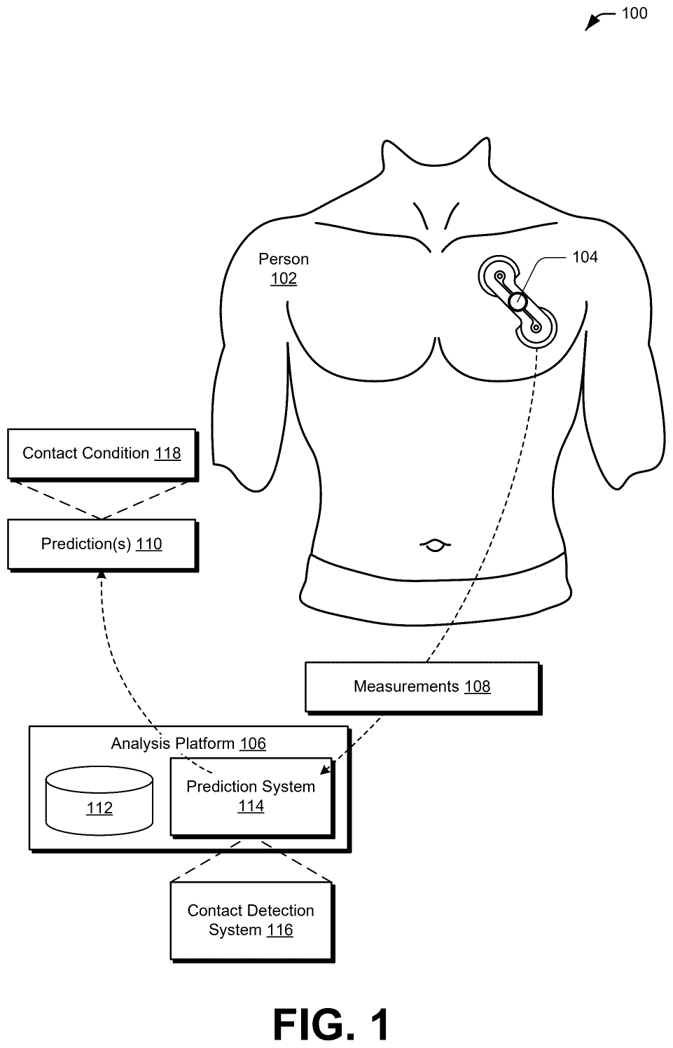

FIG. 1 is a block diagram of a non-limiting example 100 of an environment that is operable to employ techniques for conduction-based contact detection for physiological sensors as described herein. The illustrated example 100 includes person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. In some scenarios, for instance, the monitoring device 104 may be provided to record electrical activity of the person 102's heart over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. In at least one example, the monitoring device 104 is provided to record photoplethysmography (“PPG”) data over an observation period, such as to collect blood oxygen saturation (“SpO2”) data. Alternatively or in addition, the monitoring device 104 may be used to output measurements 108 (e.g., a time sequence of measurements such as a time sequence of electric potential measurements), which may indicate an observation or be used to generate a prediction of one or more events.

In connection with the monitoring device, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternately or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

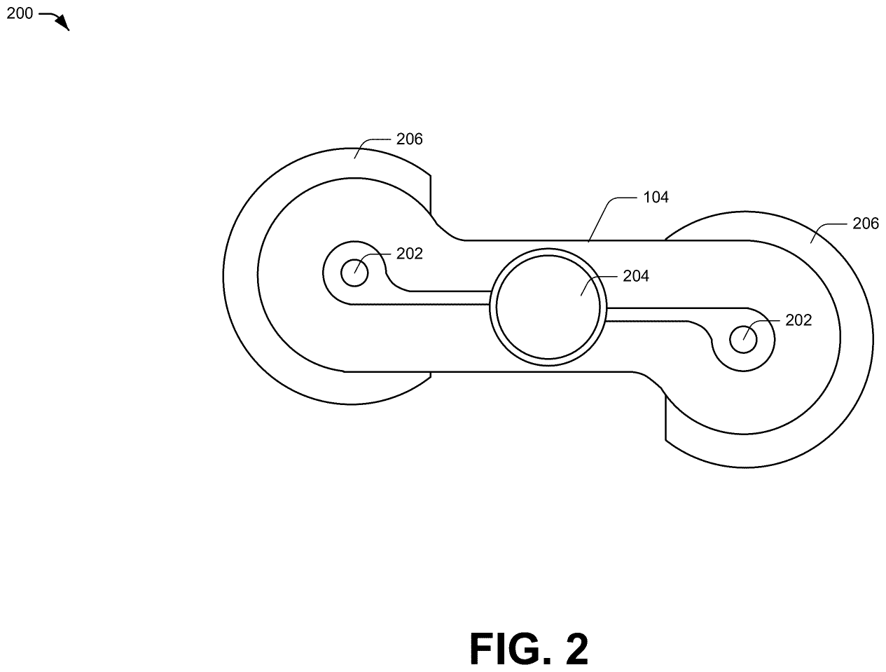

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner as monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively or additionally, the monitoring device 104 may have a similar form factor as for clinical settings, but have different functionality, such as functionality that prevents a wearer from viewing the measurements.

In one or more implementations, the monitoring device 104 may be configured to offload measurements during the course of the observation period. By way of example, the monitoring device 104 may offload the measurements by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of physiological conditions.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device's storage.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternately or additionally be configured to offload the measurements 108 over one or more wireless connections.

Once the monitoring device 104 produces the measurements 108, the measurements can be provided to the analysis platform 106. As noted above, the measurements 108 are communicated to the analysis platform 106 over wired and/or wireless connection(s).

In scenarios where the analysis platform 106 is implemented partially or entirely on the monitoring device 104, for instance, the measurements 108 may be transferred over a bus from the device's local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured generate one or more predictions 110. For example, the measurements 108 and/or additional measurements may be processed by a smartphone associated with the user, a smartphone or other dedicated device associated with the monitoring device 104, and/or one or more server computers at a data center or other location that can be utilized by an entity associated with the monitoring device 104, to name just a few. In other words, those other devices may implement at least a portion of the analysis platform 106 and/or a prediction system 114.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternately or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth (e.g., Bluetooth Low Energy links), near-field communication (NFC), Long Term Evolution (LTE) standards such as 5G, and so forth. Monitoring devices 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc.

In one or more implementations, the analysis platform 106 may be implemented in whole or in part at the monitoring device 104. Alternately or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes storage device 112. In accordance with the described techniques, the storage device 112 is configured to maintain the measurements 108 and/or other measurements or information processed by the prediction system 114 to generate one or more predictions 110. The storage device 112 may represent one or more databases and also other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the analysis platform 106 also includes the prediction system 114. The prediction system 114 represents functionality to process the measurements 108 to generate the one or more prediction(s) 110. Alternatively or in addition, the prediction system 114 may output one or more time sequences indicating an observation or prediction of one or more events, over time. It is also to be appreciated that the prediction system 114 may output different combinations of multiple predictions in variations.

In at least one implementation, the prediction system 114 uses machine learning to generate one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively or additionally, the prediction system 114 may include logic (a machine learning model and/or other types of logic) to pre-process the obtained measurements, such as to extract various cardiovascular and/or other physiological or non-physiological features from the sequences of measurements. The illustrated example 100 also includes prediction(s) 110, which corresponds to the output of the prediction system 114.

In various examples, the prediction system 114 is representative of and/or includes a contact detection system 116 and the prediction 110 includes and/or is representative of a contact condition 118. For instance, as further described in more detail below the contact detection system 116 is operable to implement conduction-based techniques to generate the contact condition 118. The contact condition 118, for instance, indicates whether one or more components of the monitoring device 104 are in sufficient contact with a skin surface of the person 102, such as sufficient to support collection of the measurements 108.

FIG. 2 depicts a non-limiting example 200 of a monitoring device. The illustrated example 200, for instance, depicts the monitoring device 104.

In accordance with the described techniques, the monitoring device 104 includes one or more sensors 202, examples of which include but are not limited to one or more pairs of electrodes, a PPG sensor, one or more other optical sensors, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102's heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques, such as but not limited to a watch, patch, headband, strap, and so forth.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the person 102's heart's electrical activity and/or changes in blood volume. In one or more implementations, the processor further generates one or more communicable packages of data that include one or more of the measurements 108 and/or other measurements, such as accelerometer data and oxygen saturation (SpO2) measurements. Alternately or additionally, the processor produces and/or causes storage of other data, which may be used for predicting classifications of physiological conditions, e.g., sleep apnea.

In implementations where the monitoring device 104 is configured for wireless transmission, the transmitter 204 may transmit the measurements wirelessly as a stream of data to a computing device. In one or more implementations, for instance, the monitoring device 104 is configured to transfer (e.g., transmit and/or receive) information (e.g., electrical potential measurements) via a Bluetooth Low Energy (BLE) connection. Alternately or additionally, the monitoring device 104 may buffer the measurements (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Conduction-Based Contact Detection for Physiological Sensors

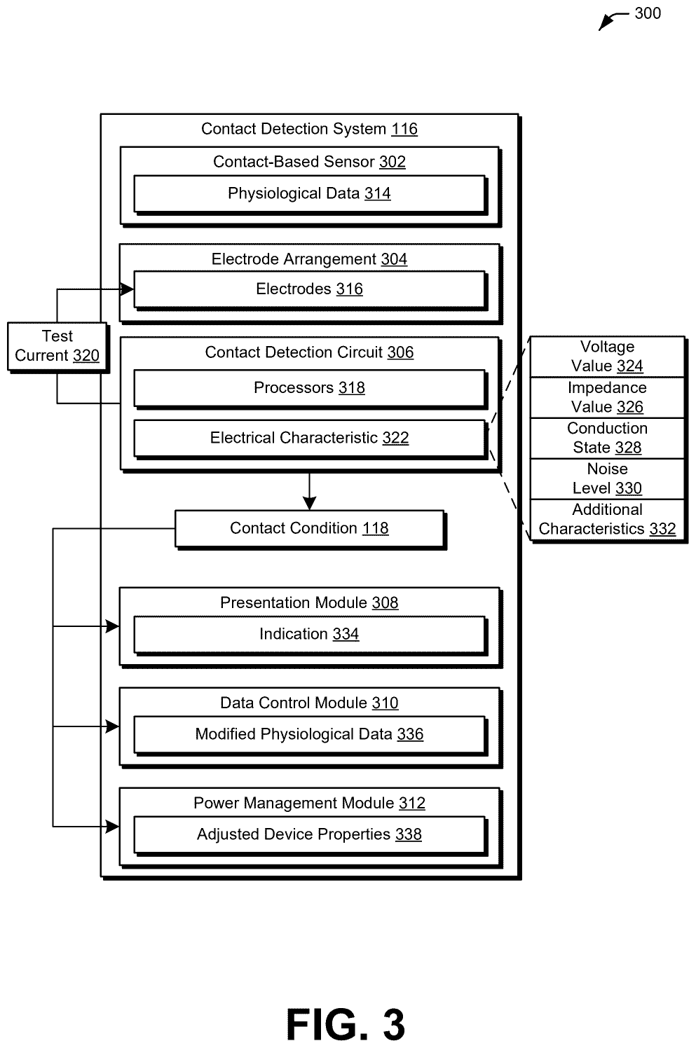

FIG. 3 depicts a non-limiting system in an example implementation 300 of conduction-based contact detection for physiological sensors showing operation of the contact detection system 116 of FIG. 1 in more detail. In various examples, the contact detection system 116 is representative of, supports functionality of, is implementable by, and/or includes (either partially or wholly) a wearable device, such as the monitoring device 104.

In the illustrated example, the contact detection system 116 includes a contact-based sensor 302, an electrode arrangement 304, and a contact detection circuit 306 that are operable to generate and/or determine a contact condition 118 of the contact-based sensor 302. The contact detection system 116 is further illustrated to include a presentation module 308, a data control module 310, and a power management module 312 that are operable to perform a variety of functionality based on the contact condition 118 as described in the following discussion.

To begin in this example, the contact-based sensor 302 of the contact condition 118 is configured to collect physiological data 314 of a user, e.g., the person 102. In one or more examples, the contact-based sensor 302 is and/or includes one or more properties of the sensors 202 discussed above with respect to FIG. 2. A variety of types of contact-based sensor 302 are considered to collect a variety of physiological data 314.

In at least one example, the contact-based sensor 302 includes a photoplethysmography (PPG) sensor. The PPG sensor may include one or more light-emitting components (e.g., light-emitting diodes) and/or one or more light-detecting components (e.g., photodiodes) configured to emit light into and detect light reflected from the skin surface of the user. The PPG sensor may collect data related to blood volume changes in a microvascular tissue bed, which can be used to derive physiological parameters such as heart rate, blood oxygen saturation (SpO2), respiration rate, blood pressure, and so forth. In various examples, accuracy of the PPG sensor is partially or wholly based on sufficient contact with the skin surface.

This is by way of example and not limitation, and the contact-based sensor 302 may be configured in various ways. For instance, the contact-based sensor 302 may include one or more of a temperature sensor configured to measure skin temperature, a heat flux sensor configured to measure heat transfer between the skin surface and the environment, a galvanic skin response sensor configured to measure skin conductance, a bioimpedance sensor configured to measure tissue impedance, and so forth. Accordingly, the physiological data 314 may include various types of measurements such as heart rate data, blood oxygen levels, skin temperature readings, perspiration levels, electrical conductivity of the skin, tissue impedance values, respiration rate, blood pressure measurements, and/or other biometric information collected by the contact-based sensor 302.

The contact detection system 116 further includes the electrode arrangement 304, which is illustrated to include one or more electrodes 316, e.g., biopotential electrodes. The electrode arrangement 304, for instance, is configured to establish a conduction path with the skin surface (and/or subdermal tissue) of the user, such as when the contact-based sensor 302 is properly positioned. The electrode arrangement 304 may include various numbers and/or configurations of electrodes 316, such as a single electrode positioned adjacent to the contact-based sensor 302, a pair of electrodes positioned on opposite sides of the contact-based sensor 302, or multiple electrodes arranged in a pattern surrounding the contact-based sensor 302.

A variety of suitable materials and/or dimensions of the electrodes 316 are considered. For instance, the electrodes 316 may include one or more biocompatible conductive materials (e.g., silver, silver chloride, gold, etc.) suitable for skin contact. The electrodes 316 may have various dimensions, such as circular electrodes with diameters ranging from 1 mm to 20 mm, or rectangular electrodes with lengths and widths in the range of 2 mm to 30 mm. The dimensions of the electrodes 316 may be selected based on factors such as an overall size of the monitoring device 104, a particular application, user dimensions and/or demographics, a desired signal quality, a spatial relationship to the contact-based sensor 302, and so forth.

In various examples, the electrode arrangement 304 includes at least one electrode 316 positioned proximate to the contact-based sensor 302, e.g., a proximate electrode, such that contact of the proximate electrode with the skin surface is correlated to contact of the contact-based sensor 302 with the skin surface. For instance, a proximate electrode is positioned sufficiently adjacent to the contact-based sensor 302 such that both components experience substantially similar contact conditions with the skin surface. In some implementations, a proximate electrode is positioned within a range of 0.1 mm to 10 mm from the contact-based sensor 302, however other distances may be used such as based on a particular device configuration, sensor type, electrode arrangement 304, and so forth.

In some implementations, the electrodes 316 may be categorized as driven electrodes and/or passive electrodes. A driven electrode, for instance, is an electrode that is operable to apply an electrical signal (e.g., voltage, current, etc.) to a conduction path while a passive electrode is operable to detect and/or receive an electrical signal. In various examples, a driven electrode may include a right-leg drive (“RLD”) electrode, e.g., an electrode used as part of an ECG system, and/or a bioimpedance drive electrode, e.g., an electrode used in a bioimpedance measurement system.

In some examples, one or more of the electrodes 316 are integrated into a measurement system to collect data related to another physiological parameter different than what is measured by the contact-based sensor 302. For example, the electrodes 316 may be part of an electrocardiogram (ECG) measurement system of a wearable device that monitors cardiac electrical activity, while the contact-based sensor 302 is a PPG sensor to measure blood oxygen levels. This integration allows the contact detection system 116 to leverage existing electrode infrastructure for a primary physiological measurement function and a secondary function of contact detection, thereby improving efficiency and reducing component redundancy.

In at least one example, one or more of the electrodes 316 are configured as biopotential electrodes designed to interface with a biopotential amplifier. A biopotential electrode, for instance, may be used to detect relatively minute electrical signals (e.g., in a range of 0.5 to 5 millivolts) generated by physiological processes, such as cardiac activity or muscle contractions. In some implementations, the electrodes 316 may be connected to high-impedance, low-noise amplifiers to enhance detection of relatively weak bioelectric signals. The biopotential amplifiers may include differential amplification stages such as to reject common-mode noise and/or improve a signal-to-noise ratio of the detected physiological signals.

The contact detection system 116 further includes the contact detection circuit 306. The contact detection circuit 306, for instance, is coupled to the electrode arrangement 304 to leverage functionality of and/or to support operation of the one or more electrodes 316. The contact detection circuit 306 is further configurable to monitor and analyze a conduction path between the electrode arrangement 304 and an adjacent surface (e.g., a skin surface of a user) such as to determine the contact condition 118 of the contact-based sensor 302.

The contact detection circuit 306 may include various hardware/software components or elements such as one or more signal generators, amplifiers, filters, memory components, multiplexers, regulators, microcontrollers, connections, analog-to-digital converters, comparators, processing elements, etc., that interact to perform a variety of functionality. In various examples, the contact detection circuit 306 includes components particular to biological monitoring in wearable devices, such as one or more calibration modules to adjust for variations in skin conductivity, temperature compensation circuits to account for environmental factors, adaptive thresholding algorithms to dynamically adjust contact detection sensitivity (e.g., based on real-time measurements and historical data patterns) and so forth. This is by way of example and not limitation, and various components of the contact detection circuit 306 are considered.

In the illustrated example, the contact detection circuit 306 includes one or more processors 318 that are configurable to determine a contact condition 118 of the contact-based sensor 302. For instance, the contact detection circuit 306 leverages the processors 318 to determine the contact condition 118 based on an electrical characteristic 322 of a conduction path between one or more electrodes 316 of the electrode arrangement 304 and a skin surface of the user. A variety of electrical characteristics 322 are considered, such as but not limited to one or more of a voltage value 324, an impedance value 326, a conduction state 328 (e.g., conduction, nonconductive, partially conductive, etc.) a noise level 330, or various additional characteristics 332.

For instance, the voltage value 324 may represent a potential difference measured across the conduction path, which can indicate a quality of electrical contact between the electrodes and the skin surface. In various examples, the contact detection circuit 306 leverages one or more comparators to detect the voltage value 324. The impedance value 326 may reflect a resistance to current flow through the conduction path, with a relatively lower impedance corresponding to enhanced contact.

The conduction state 328 may categorize electrical connectivity, ranging from fully conductive to non-conductive. The noise level 330 may quantify an amount of signal fluctuation, which can be indicative of poor contact or interference. Additional characteristics 332 may include factors such as signal stability over time, frequency response of the conduction path, or phase shifts in applied alternating currents, which may provide further insights into the contact quality and skin-electrode interface properties.

In an example to determine the contact condition 118, the contact detection circuit 306 injects a test current 320 through at least one of the electrodes 316, e.g., a driven electrode. The test current 320, for instance, represents an electrical signal applied to the electrode arrangement 304 to evaluate the conduction path, and may include alternating current (AC), direct current (DC), pulsed signals, signals of various frequencies (e.g., 1 kHz to 100 kHz), amplitudes (e.g., 10 nA to 1 mA), waveforms (e.g., sine, square, triangular), or other electrical characteristics suitable for bioimpedance measurements. The contact detection circuit 306 is operable to measure an electrical characteristic 322, such as a voltage value 324, required to drive the test current 320 through a conduction path that includes the driven electrode, the skin surface, and an additional electrode, e.g., a passive electrode. Based on the electrical characteristic 322, the contact detection circuit 306 determines the contact condition 118.

For instance, a relatively high voltage value 324 corresponds to high resistance present in the conduction path, suggesting insufficient contact between the electrode and the skin surface and therefore insufficient contact of the contact-based sensor 302. Conversely, a relatively low voltage value 324 corresponds to a low-resistance conduction path, which indicates proper adherence of the electrode to the skin surface and therefore sufficient contact of the contact-based sensor 302.

In some examples, the contact detection circuit 306 implements a threshold to determine the contact condition 118. For instance, if the electrical characteristic 322 (e.g., a voltage value 324) exceeds a predetermined threshold, the contact detection circuit 306 may determine that the contact condition 118 indicates insufficient contact between the contact-based sensor 302 and the skin surface. If the electrical characteristic 322 is below the threshold, the contact detection circuit 306 may determine that the contact condition 118 indicates sufficient contact between the contact-based sensor 302 and the skin surface.

In an alternative or additional example, the contact condition 118 includes a quantification of an amount of contact between the contact-based sensor 302 and the skin surface based on the detected electrical characteristic 322. The quantification may be expressed in various ways, such as a percentage of contact area, a numerical score on a predefined scale (e.g., 1-10), a percentage, a categorical assessment (e.g., “excellent,” “good,” “fair,” or “poor”), and so forth. By way of example, the contact detection circuit 306 may implement one or more algorithms that correlate ranges of values of the electrical characteristics 322 to corresponding contact quality levels, such as to provide granular assessment of the sensor-to-skin interface. For instance, a relatively low impedance value may correspond to an “excellent” contact condition, while progressively higher impedance values may indicate “good,” “fair,” or “poor” contact conditions.

The contact detection circuit 306 and/or processors 318 can implement a variety of techniques and/or leverage various modalities as part of determining the contact condition 118. For example, the contact detection circuit 306 may employ one or more adaptive filtering algorithms, such as to compensate for variations in skin conductivity due to perspiration, temperature changes, individual physiological differences, etc. Additionally or alternatively, the processors 318 may leverage one or machine learning techniques, such as to implement a machine learning model trained on historical contact data to improve detection accuracy over time by recognizing user-specific patterns in the electrical characteristics 322.

In various embodiments, the contact condition 118 can include information in addition or alternative to a contact quality assessment. For example, the contact condition 118 may include a confidence score that indicates a reliability of the physiological data 314 being collected, a temporal prediction of “how long” the current contact condition 118 will persist based on historical patterns, a spatial mapping of contact quality across different regions of the sensor-skin interface, an indication of changes to the contact condition 118 over time, and so forth. Additionally or alternatively, the contact condition 118 can include diagnostic information about a particular cause of poor contact, such as whether a contact issue is caused by excessive movement, perspiration, dry skin, device positioning, etc. Accordingly, the contact detection system 116 is operable to perform a variety of functionality based on the information included in the contact condition 118.

For instance, the presentation module 308, data control module 310, and power management module 312 of the contact detection system 116 are implementable to perform various actions based on the determined contact condition 118. In various examples, the presentation module 308 is implemented to generate an indication 334 of the contact condition 118. The indication 334 may include visual notifications, audio alerts, or haptic feedback to inform the user about the contact status of the contact-based sensor 302. The presentation module 308 is further operable to cause output of the indication 334, such as via the wearable device and/or one or more interconnected devices.

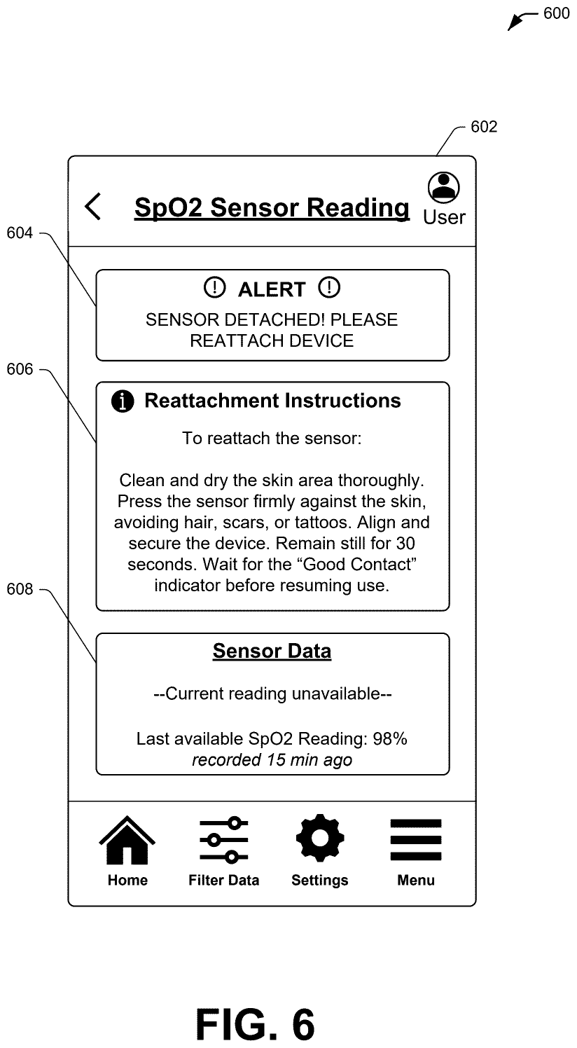

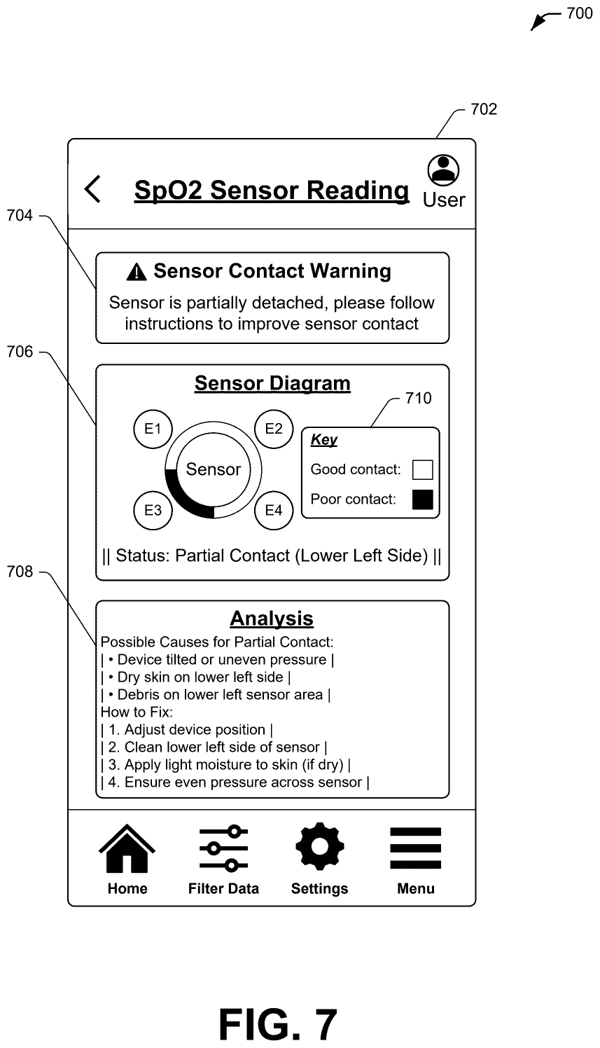

The presentation module 308 may generate various types of indications 334 to convey the contact condition 118. For example, the indication 334 may include a visual notification displayed on a screen of the wearable device, such as a color-coded icon (e.g., green for good contact, yellow for partial contact, red for poor contact) or a textual message describing the contact condition 118. As further discussed below with respect to FIGS. 6 and 7, in various examples the indication 334 may include diagnostic information about a cause of the contact condition 118 and/or user guidance to improve contact between the contact-based sensor 302 and the skin surface.

In some implementations, the indication 334 may include an audio alert, such as a series of beeps or a spoken message. The presentation module 308 may also generate haptic feedback, such as vibration patterns of varying intensity or duration that correspond to changes in the contact condition 118. In some examples, the presentation module 308 may transmit the indication 334 to an associated device, such as a smartphone or tablet, for presentation. The presentation module 308 may also adjust a frequency or intensity of the indications 334 such as based on a severity of the contact issue and/or user preferences.

The data control module 310 is operable to analyze the physiological data 314 based on the contact condition 118, such as to generate modified physiological data 336. For example, the data control module 310 may weight, filter, or flag portions of the physiological data 314 that correspond to periods of insufficient or reduced contact. In this way, the contact detection system 116 is able to provide informed analysis of physiological data 314 that considers a contact status of the contact-based sensor 302 in real time and during post-wear analysis.

By way of example, consider a scenario in which a smartwatch that includes a PPG sensor is worn by a user during exercise, and the PPG sensor becomes disengaged from the skin surface of the user during activity. Using conventional techniques, the PPG sensor continues to collect PPG data which causes inaccurate calculations related to heart rate and/or blood oxygenation. However, using the techniques described herein, the data control module 310 can identify instances of insufficient sensor contact and exclude particular data from analysis, apply one or more correction factors based on a degree of contact loss, or flag the data with confidence scores that downstream applications can use during data analysis. This prevents propagation of measurement errors and ensures that health insights are based on reliable modified physiological data 336.

Additionally or alternatively, the contact condition 118 can leverage the power management module 312 to implement adjusted device properties 338 based on the contact condition 118. The adjusted device properties 338, for instance, can include modifications to power consumption settings, sensor activation states, and/or data collection frequencies. In various implementations, the power management module 312 may pause, adjust, terminate, and/or initiate data collection or power consumption of the contact-based sensor 302 based on the contact condition 118.

For example, when the contact condition 118 indicates insufficient contact between the contact-based sensor 302 and the skin surface, the power management module 312 may temporarily suspend data collection operations of the contact-based sensor 302 such as to conserve battery power and computational resources. Conversely, when the contact condition 118 indicates sufficient contact has been restored, the power management module 312 may automatically resume data collection at a default sampling rate.

In implementations in which the contact condition 118 includes a quantification of contact quality, the power management module 312 may proportionally adjust properties of the contact-based sensor 302, such as sampling frequency, LED intensity for a PPG sensor, or power allocation, based on a degree of contact detected. For instance, with 75% contact quality, the power management module 312 can reduce sampling frequency by 25% and maintain particular monitoring functions.

In additional or alternative examples, the power management module 312 may dynamically adjust properties, e.g., an intensity or frequency, of the test current 320 based on the detected contact condition 118. For instance, the power management module 312 may detect an interval of “sufficient contact” that exceeds a temporal threshold and accordingly reduce an intensity and/or frequency of the test current 320 such as to conserve battery life of a wearable device. Alternatively or additionally, the power management module 312 may automatically increase the frequency and/or intensity of the test current 320 responsive to detection of reduction and/or degradation of contact quality, such as to improve measurement accuracy and/or frequency during intervals with dynamic contact conditions 118.

Accordingly, the techniques described herein provide a robust and reliable approach to detect and respond to contact conditions 118 in wearable devices that include physiological sensors. By leveraging conduction-based techniques, the contact detection system 116 are operable to enhance accuracy and reliability of physiological measurements, optimize power consumption, and improve a user experience. The following examples illustrate various implementations of the contact detection system and user interfaces that may be employed to convey contact condition information to users.

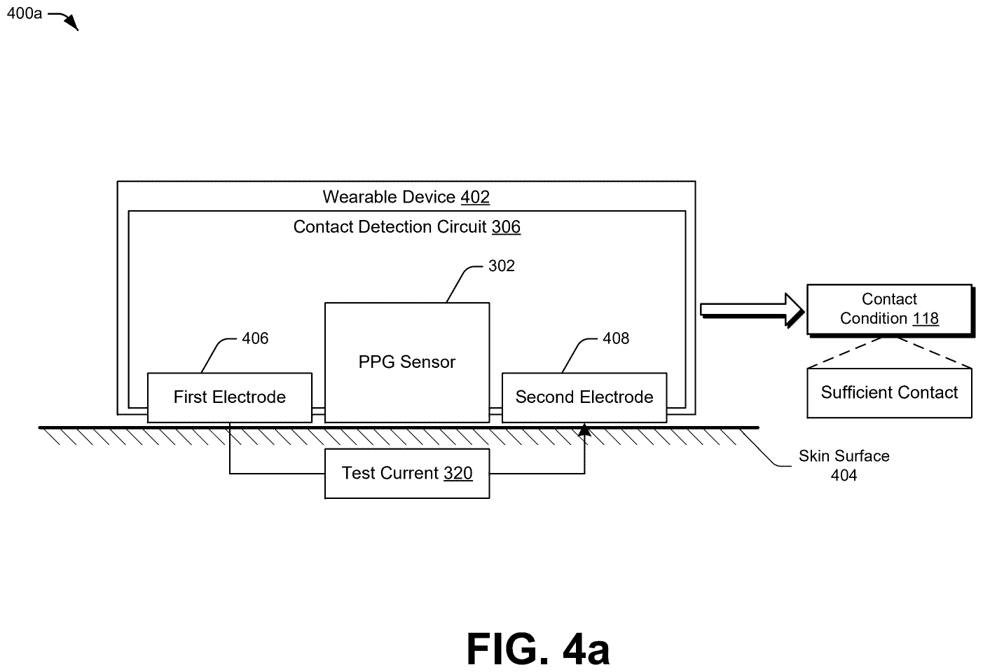

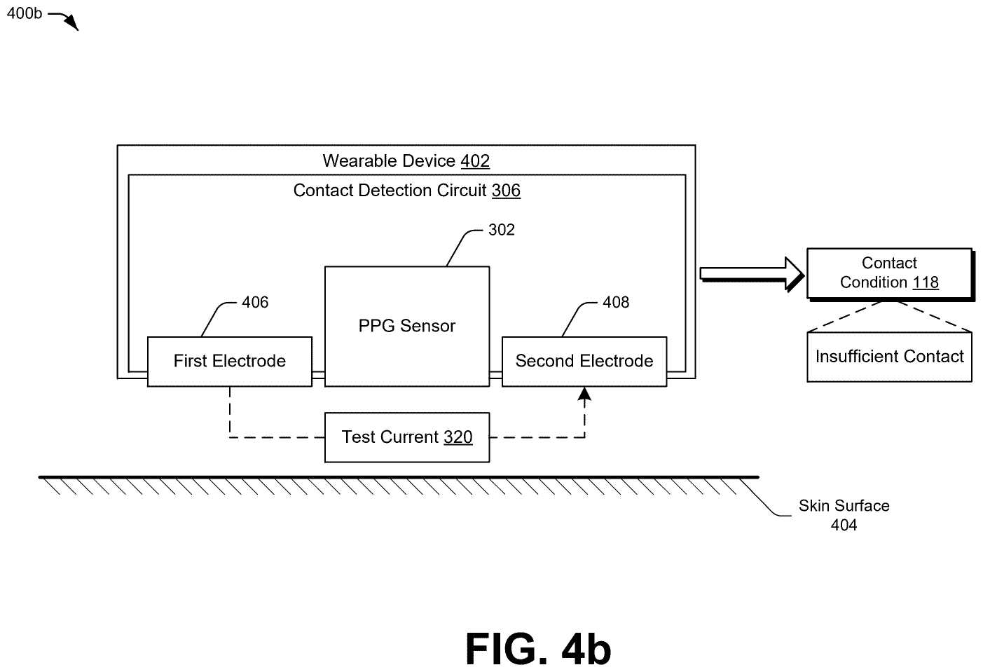

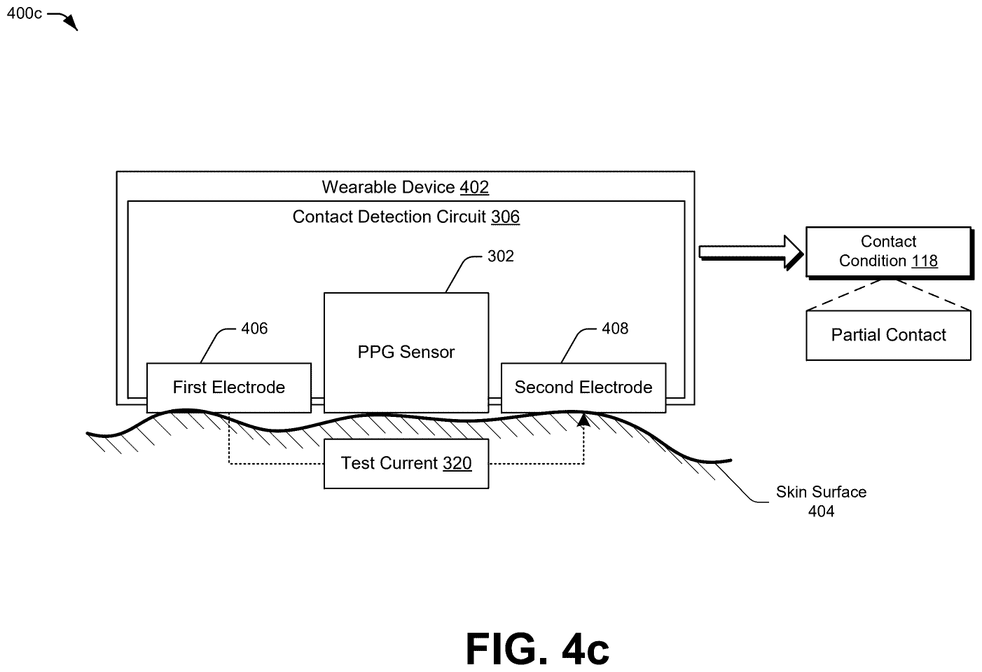

FIGS. 4a, 4b, and 4c depict nonlimiting examples 400a, 400b, and 400c of conduction-based contact detection for physiological sensors in which various contact conditions are detected by a wearable device.

For instance, as shown in FIG. 4a a wearable device 402 that includes a contact-based sensor 302 and a contact detection circuit 306 is attached to a skin surface 404 of a user. The wearable device 402, for instance, can be implemented in various form factors such as a smartwatch, fitness band, medical patch, ring, etc. In this example, the contact-based sensor 302 includes a photoplethysmography (PPG) sensor configured to collect physiological data 314 from a user. The contact detection circuit 306 is coupled to an electrode arrangement 304, which in the illustrated example includes a first electrode 406 and a second electrode 408.

The first electrode 406 and the second electrode 408 are positioned proximate to the contact-based sensor 302. For instance, the first electrode 406 is positioned adjacent to a first side of the contact-based sensor 302, while the second electrode 408 is positioned adjacent to a second side of the contact-based sensor 302. The first electrode 406 and the second electrode 408 are positioned such that contact of the electrodes with the skin surface 404 correlates with contact of the contact-based sensor 302 with the skin surface 404.

In accordance with the techniques described above, the contact detection circuit 306 determines a contact condition 118 that indicates whether the contact-based sensor 302 has sufficient contact with the skin surface 404 to collect reliable physiological data 314. To do so, the contact detection circuit 306 injects a test current 320 through the first electrode 406 to establish a conduction path through the skin surface 404 to the second electrode 408. For instance, the contact detection circuit 306 detects an electrical characteristic 322, e.g., a voltage value 324, used to drive the test current 320 and determines the contact condition 118 based on the electrical characteristic 322.

In this example, the contact detection circuit 306 determines that the voltage value 324 used to drive the test current 320 from the first electrode 406 through the skin surface 404 to the second electrode 408 is below a threshold which corresponds to relatively low impedance in the conduction path. The low impedance indicates that the first electrode 406 and second electrode 408 are in contact with the skin surface 404, which is relatively conductive. Accordingly, because of the proximity of the electrodes to the contact-based sensor 302, the contact detection circuit 306 generates the contact condition 118 to indicate sufficient contact between the contact-based sensor 302 and the skin surface 404 to collect reliable physiological data 314.

FIG. 4b illustrates an example 400b in which the wearable device 402 is separated from the skin surface 404. The wearable device 402 may become disengaged from the user for various reasons, such as movement during physical activity, adhesive failure, intentional removal of the wearable device 402, displacement when adjusting clothing or accessories, foreign object interference, and so forth. Similar to the example 400a, in this example 400b the contact detection circuit 306 determines a contact condition 118 by injecting a test current 320 through the first electrode 406 and measuring an electrical characteristic 322 such as a voltage value 324 used to drive the test current 320.

However, due to the separation between the wearable device 402 and the skin surface 404, the conduction path is interrupted by an air gap with significantly lower electrical conductivity and higher resistance relative to skin tissue. Based on an electrical characteristic 322 of the interrupted conduction path, e.g., a voltage value 324 above a threshold, the contact detection circuit 306 determines a contact condition 118 that indicates insufficient contact between the contact-based sensor 302 and the skin surface 404. This detection enables the wearable device 402 to identify when the contact-based sensor 302 is not properly positioned to collect reliable physiological data 314.

FIG. 4c illustrates an example 400c in which the wearable device 402 maintains partial contact with the skin surface 404. In various implementations, the skin surface 404 may not present a flat, uniform interface due to various factors such as user movement during physical activity, varying hydration conditions of the skin, presence of hair, skin texture variations, or anatomical contours at the wearing location. These conditions can create inconsistent contact between the wearable device 402 and the skin surface 404, resulting in a partial contact scenario.

Similar to the examples 400a and 400b, in this example 400c the contact detection circuit 306 determines a contact condition 118 by injecting one or more test currents 320 through the first electrode 406 and measuring an electrical characteristic 322 of the conduction path. In this example, the contact detection circuit 306 generates the contact condition 118 to indicate that the contact-based sensor 302 maintains partial contact with the skin surface 404 based on the electrical characteristic 322. The contact detection circuit 306 is able to detect the partial contact in various ways.

For instance, in the partial contact scenario, the electrical characteristic 322 may exhibit intermediate values that fall between those associated with full contact and no contact. By way of example, a “full contact” scenario exhibits an impedance value of approximately 10 kΩ and a “no-contact” scenario exhibits an impedance value of approximately 1 MΩ, while a partial contact scenario may exhibit an intermediate impedance value of 100 kΩ. Thus, a detected intermediate value may indicate that a portion of an electrode is in contact with the skin surface 404, while an alternative portion of the electrode is not in contact with the skin surface 404.

Additionally or alternatively, the contact detection circuit 306 may determine the partial contact scenario based on measured fluctuations in the electrical characteristic 322 over a temporal period. For instance, the contact detection circuit 306 may collect multiple values of the electrical characteristic 322 over a defined time span, such as by injecting multiple test currents 320 at a particular rate. Based on characteristics of and/or a relationship between the multiple values (e.g., a degree of variance between values, frequency distribution patterns, temporal stability, rate of change between consecutive values, correlation with user movement patterns, etc.) the contact detection circuit 306 is operable to detect the partial contact scenario.

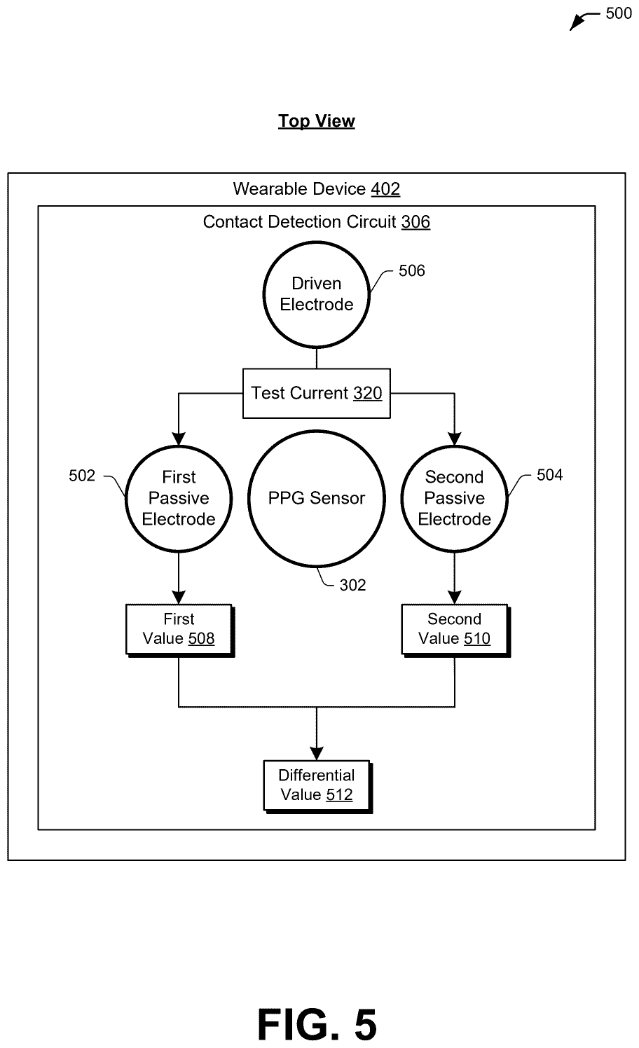

As described below in more detail with respect to FIG. 5, in some examples the partial contact scenario is detectable using an electrode arrangement 304 that includes three or more electrodes 316. For instance, the contact detection circuit 306 can measure and compare electrical characteristic 322 for multiple conduction paths between different pairs of electrodes. The contact detection circuit 306 can further calculate differential values between these measurements to detect asymmetrical contact patterns and localized areas of poor contact, which contribute to a determination of a partial contact scenario.

Accordingly, the contact detection circuit 306 leverages one or more of the above-described techniques to generates a contact condition 118 that indicates a partial contact state of the contact-based sensor 302. The wearable device 402 can perform various functionality responsive to detection of the partial contact state. For example, the wearable device 402 may apply proportional correction factors to the physiological data 314 based on the degree of contact, adjust sampling rates or sensor sensitivity to compensate for the partial contact, provide user guidance to address the particular contact issue (such as instructions to tighten the device or moisten the skin), and/or assign confidence scores to the collected data that reflect the partial contact state. Additionally or alternatively, the wearable device 402 may implement power management strategies based on the partial contact state, such as to reduce sampling frequency of the contact-based sensor 302 at a rate proportional to the quality of the partial contact detected.

FIG. 5 depicts a nonlimiting example 500 of conduction-based contact detection for physiological sensors in which an electrode arrangement supports determination of multiple electrical conditions.

In this example 500, a wearable device 402 that includes a contact detection circuit 306 is depicted from the top view. The contact detection circuit 306 includes an electrode arrangement 304 that includes three electrodes 316, such as a first passive electrode 502, a second passive electrode 504, and a driven electrode 506. One or more of the first passive electrode 502, second passive electrode 504, and/or driven electrode 506 are positioned proximate to a contact-based sensor 302, e.g., a PPG sensor, such that a contact status of the proximate electrodes corresponds to a contact status of the contact-based sensor 302.

In this example, the contact detection circuit 306 is operable to inject a test current 320 through the driven electrode 506, which can flow through a conduction path that includes one or more of the first passive electrode 502, the second passive electrode 504, and/or an adjacent surface or medium. Accordingly, the contact detection circuit 306 may detect a first value 508 of an electrical characteristic 322 of a conduction path between the driven electrode 506, a skin surface, and the first passive electrode 502. The contact detection circuit 306 may further detect a second value 510 of an electrical characteristic 322 of a conduction path between the driven electrode 506, the skin surface, and the second passive electrode 504.

The contact detection circuit 306 may leverage the first value 508 and second value 510 to determine a differential value 512, which can inform various functionality. The differential value 512, for instance, represents a calculated difference between the first value 508 and the second value 510, and thus can indicate whether the contact-based sensor 302 has asymmetrical contact with the skin surface. For example, a relatively low differential value 512 indicates similar contact status of the first passive electrode 502 and the second passive electrode 504. However, a significant differential value 512 (e.g., a differential value 512 above a threshold) may indicate partial contact of the contact-based sensor 302. For instance, the contact detection circuit 306 generates a significant differential value 512 in an example in which a first side of the contact-based sensor 302 maintains sufficient contact while an opposite side of the contact-based sensor 302 is partially or wholly detached. Accordingly, the differential value 512 can inform detection of sensor tilting, uneven pressure, or partial lifting of the contact-based sensor 302 from the skin surface.

In at least one example, the contact detection circuit 306 is configurable to inject multiple test currents 320 across a range of frequencies through the driven electrode 506. For instance, the contact detection circuit 306 may generate an impedance spectrogram based on the responses detected at the first passive electrode 502 and/or the second passive electrode 504. The impedance spectrogram may provide information about the contact condition 118 between the wearable device 402 and the skin surface across various frequency ranges.

The illustrated configuration in FIG. 5 is provided by way of example and not limitation and a variety of electrode arrangements 304 are contemplated. For instance, an electrode arrangement 304 may include multiple driven electrodes and/or multiple passive electrodes arranged in various configurations around a sensor. This configuration may enable the contact detection circuit 306 to identify whether each of the multiple electrodes maintains contact with the skin surface, such as to provide granular contact mapping of the contact-based sensor 302 and identify a contact quality of discrete portions of the contact-based sensor 302. Accordingly, the electrode arrangement 304 can be configured in a variety of ways to support various functionality and use cases.

FIG. 6 depicts a nonlimiting example 600 of conduction-based contact detection for physiological sensors in which a user interface for a loss of contact scenario is shown.

In the illustrated example 600, a user interface 602 for SpO2 sensor readings displays a variety of digital content (e.g., various indications 334) responsive to a contact condition 118 that indicates that a contact-based sensor 302 has become detached from a skin surface. The user interface 602 includes various elements that provide information about the sensor contact status as well as user guidance for reattachment of the contact-based sensor 302.

For instance, an alert element 604 is depicted near a top of the user interface 602. The alert element 604 displays a prominent warning message that indicates the sensors has become detached and a directive to reattach the sensor. Below the alert element 604, an instruction element 606 provides reattachment instructions to reattach and secure the contact-based sensor 302. The instruction element 606, for instance, includes step-by-step instructions to guide a user to reestablish contact between the contact-based sensor 302 and the skin surface.

A sensor data element 608 displays sensor data information. In the illustrated example, the sensor data element 608 presents a last available SpO2 reading that was recorded before the contact-based sensor 302 became detached, along with an indication that the current reading is unavailable due to the detached state. Thus, the techniques described herein support reliable detection of sensor contact quality, provide guidance to users to take corrective actions, and ensure physiological measurements are collected when sufficient contact is maintained between the sensor and the skin surface.

FIG. 7 depicts a nonlimiting example 700 of conduction-based contact detection for physiological sensors in which a user interface for a partial contact scenario is shown.

In this example, the contact detection system 116 determines a contact condition 118 that indicates partial contact between the contact-based sensor 302 and a skin surface in accordance with the techniques described above. The user interface 702 includes various elements (e.g., various indications 334) that provide information about the sensor contact status, such as a contact warning element 704, sensor diagram element 706, and analysis element 708.

The contact warning element 704 displays an alert that indicates the contact-based sensor 302 is partially detached and prompts the user to follow instructions to improve sensor contact. Below the contact warning element 704, a sensor diagram element 706 provides a visual representation of the sensor contact status. The sensor diagram element 706 shows a central sensor surrounded by an electrode arrangement 304 that includes four electrodes, labeled E1, E2, E3, and E4.

Using the electrode arrangement 304 that includes the four electrodes, the contact detection system 116 is able to determine variable contact of different portions of the contact-based sensor 302. Accordingly, the sensor diagram element 706 includes a key 710 that denotes “good” contact areas as white regions on the diagram and “poor” contact areas as black regions on the diagram. For instance, the sensor diagram element 706 indicates poor contact of a lower left side of the contact-based sensor, such as adjacent to the electrode labeled “E3”.

The analysis element 708 includes information about potential causes of the poor contact as well as steps to resolve the issue. For instance, the analysis element 708 includes potential causes such as device tilting, dry skin, or debris, as well as corresponding fixes such as to adjust a device position, clean the sensor, apply moisture, and ensure even pressure. This diagnostic information provides a guide for a user to resolve the partial contact issue and increase efficacy of the contact-based sensor 302.

The user interfaces 602 and 702 depict examples of a how a wearable device may output indications of contact conditions 118, such as to provide users with clear visual notifications and guidance to address sensor contact issues. By collecting and presenting this information in this way, the user interfaces 602 and 702 are able to support proper contact between contact-based sensors 302 and skin surfaces, enhance a user experience, and improve reliability and continuity of physiological data collection.

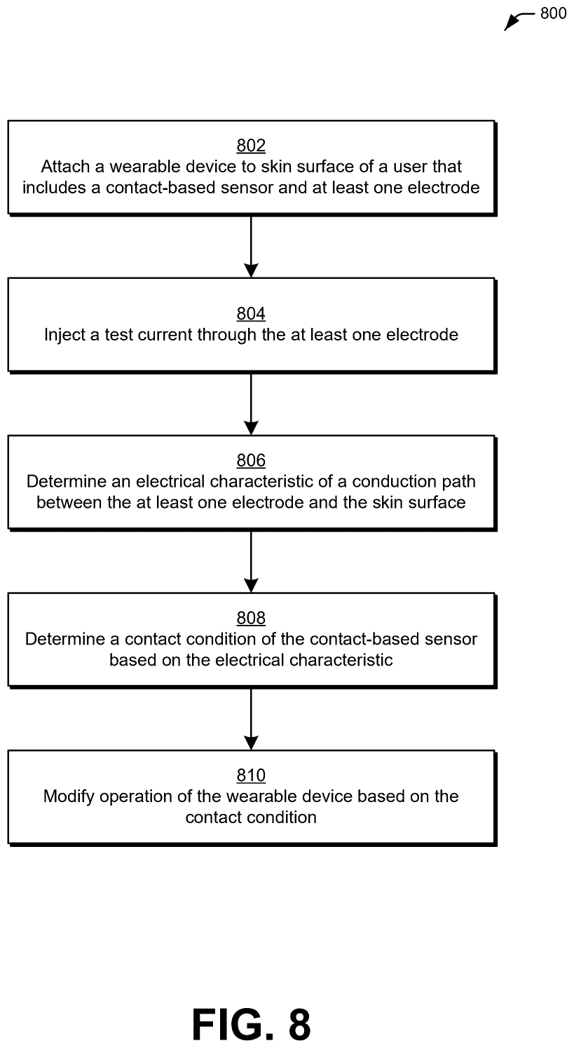

FIG. 8 depicts a flow diagram depicting an algorithm as a step-by-step procedure 800 in an example implementation, one or more steps of which are performable by a processing device to determine and respond to a contact condition of a contact-based sensor.

To begin in this example, a wearable device is attached to a skin surface of a user (block 802). The wearable device, for instance, includes a contact-based sensor 302 and at least one electrode 316. In various examples, the contact-based sensor 302 may be configured to collect physiological data 314 via contact with a skin surface of a user of the wearable device.

The electrode 316, for instance, is part of an electrode arrangement 304 that includes multiple electrodes 316. In various examples, one or more of the electrodes 316 are positioned proximal to the contact-based sensor 302 such that a contact status of the proximal electrodes corresponds to a contact status of the contact-based sensor 302.

A test current is injected through the at least one electrode (block 804). For example, the wearable device includes a contact detection circuit 306 coupled to the electrode arrangement 304. The contact detection circuit 306 is configured to inject the test current 320 through one or more electrodes 316 of the electrode arrangement 304.

An electrical characteristic of a conduction path between the at least one electrode and the skin surface is determined (block 806). The electrical characteristic 322, for instance, may include a voltage value 324 used to drive the test current 320 from a first electrode of the electrode arrangement 304 to a second electrode through a conduction path that includes one or more of the first electrode, the skin surface, and the second electrode. A variety of other electrical characteristics 322 are considered, such as an impedance value 326, conduction state 328, noise level 330, and so forth.

A contact condition of the contact-based sensor is determined based on the electrical characteristic (block 808). The contact condition 118, for instance, indicates a contact status of the contact-based sensor 302, such as whether or not the contact-based sensor 302 has sufficient contact with the skin surface to collect reliable physiological data 314. In one or more examples, the contact condition 118 includes a quantification of an amount of contact of the contact-based sensor 302 with the skin surface, such as based on a correlation to one or more properties of the electrical characteristic 322.

The operation of the wearable device is modified based on the determined contact condition (block 810). In various examples, the modification may include adjusting a data analysis performed on the physiological data 314 based on the contact condition 118. The adjustment to the data analysis may include weighting, filtering, or flagging data portions based on the contact condition 118. In some examples, the wearable device causes the contact-based sensor 302 to pause or reduce collection of the physiological data 314 responsive to a determination that the contact-based sensor is not in contact with the skin surface.

Alternatively or additionally, the operation of the wearable device is modified to output an indication 334 of the contact condition 118. The indication 334 can include visual content, audial content, and/or haptic feedback, and can convey various information such as the current contact condition 118, diagnostic information about a cause of the contact condition 118, guidance to improve contact between the contact-based sensor 302 and the skin surface, and so forth.

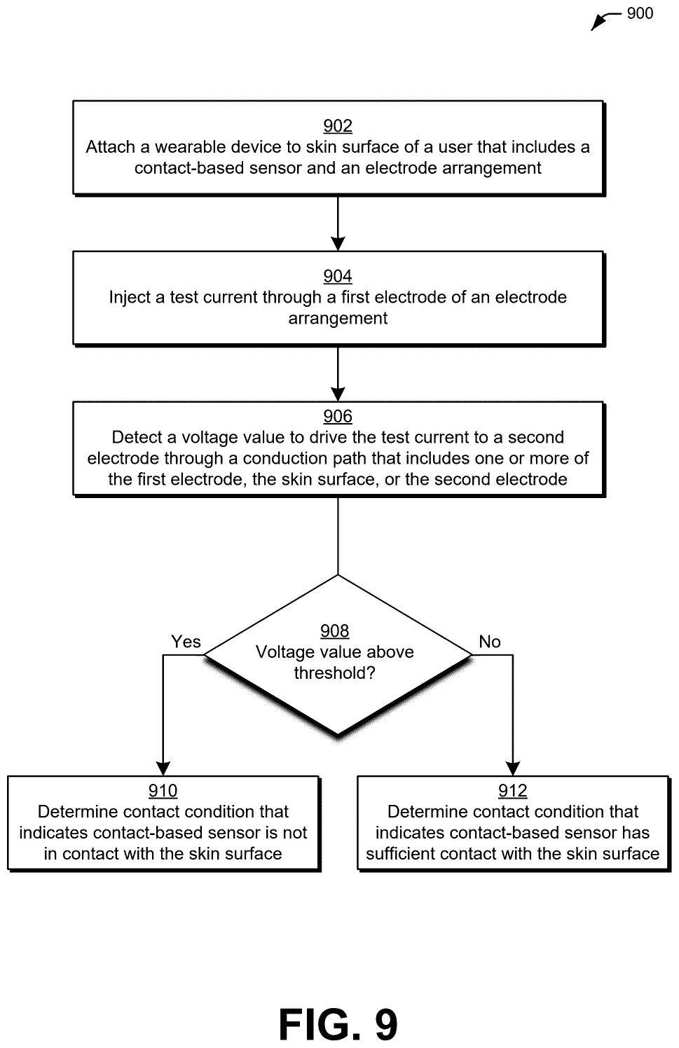

FIG. 9 illustrates a flow diagram depicting an algorithm as a step-by-step procedure 900 in an example implementation, one or more steps of which are performable by a processing device to detect contact conditions of a wearable device with a skin surface. In one or more examples, one or more steps of the procedure 900 are implementable as one or more substeps of the procedure 800.

To begin in this example, a wearable device that includes a contact-based sensor and an electrode arrangement is attached to a skin surface of a user (block 902). The contact-based sensor 302, for instance, may be configured to collect physiological data 314 via contact with the skin surface. The electrode arrangement 304 may include various electrodes 316, such as one or more driven electrodes that are operable to apply an electrical signal (e.g., voltage, current, etc.) to a conduction path and/or one or more passive electrodes that are operable to detect and/or receive an electrical signal.

A test current is injected through a first electrode of the electrode arrangement (block 904). For example, a contact detection circuit 306 of the wearable device 402 may inject the test current 320 through the first electrode, which in this example is a driven electrode. In various examples, the first electrode is positioned proximate to the contact-based sensor 302 such that a contact state of the first electrode corresponds to a contact state of the contact-based sensor 302.

A voltage value to drive the test current to a second electrode through a conduction path is detected (block 906). The conduction path, for instance, includes one or more of the first electrode, the skin surface, and/or the second electrode. The voltage value 324 corresponds to a resistance and/or impedance present in the conduction path and thus informs conductivity of the conduction path. For example, in scenarios with sufficient sensor contact, the voltage value 324 may be relatively low due to the conductivity of the skin surface. As sensor contact degrades, the voltage value 324 may increase, such as due to a relatively lower conductivity of an air gap present between the electrodes 316 and the skin surface.

A determination is made as to whether the detected voltage value is above a threshold (block 908). The threshold, for instance, represents a predetermined value that serves as a decision boundary to determine whether the contact-based sensor 302 has sufficient contact with the skin surface based on a measured electrical characteristic 322, e.g., the voltage value 324. The threshold can be based on various factors, such as user demographics, skin type and/or properties (e.g., hydration level, thickness, pigmentation, texture, lipid content, etc.) device form factor, environmental conditions, activity level, sensor type, historical measurement patterns, information collected from the contact-based sensor 302, and so forth.

In at least one example, the wearable device is operable to dynamically change the threshold, such as based on one or more of the factors listed above. By way of example, the wearable device may dynamically adjust the threshold based on physiological data 314 collected by the contact-based sensor 302, such as to increase the threshold during periods of detected high activity. In this way, the wearable device can adapt contact sensitivity based on a physiological state of a user, e.g., to compensate for increased skin conductivity due to increased perspiration to reduce an incidence of false positives.

If the voltage value is above the threshold (e.g., “Yes” at block 908), a contact condition is determined that indicates the contact-based sensor is not in contact with the skin surface (block 910). A relatively high voltage value 324 may correspond to increased resistance in the conduction path, which indicates insufficient contact between the contact-based sensor 302 and the skin surface. For instance, the elevated voltage value 324 may result from air gaps or other non-conductive materials present between the electrodes 316 and the skin surface which causes insufficient sensor contact.

If the voltage value is not above the threshold (e.g., “No” at block 908), a contact condition is determined that indicates the contact-based sensor 302 has sufficient contact with the skin surface (block 912). A relatively low voltage value 324 may correspond to relatively low resistance in the conduction path, which indicates proper electrical contact between the electrodes 316 and the skin surface. Because of the proximity of the electrodes to the contact-based sensor 302, this proper electrical contact indicates that the contact-based sensor 302 also has sufficient contact with the skin surface to collect reliable physiological data 314.

In some examples, the contact condition 118 may include a quantification of an amount of contact based on a correlation to the voltage value 324. For instance, the wearable device is operable to detect that the contact-based sensor 302 has partial contact with the skin surface. The wearable device is operable to perform a variety of functionality based on the quantification included in the contact condition 118, as described in more detail above.

Accordingly, the techniques described herein provide various advantages for wearable devices that incorporate contact-based sensors that are not possible using conventional sensor contact detection approaches. By leveraging conduction-based contact detection, devices that implement these techniques can achieve reliable and accurate physiological measurements. The ability to dynamically assess sensor contact quality in real-time allows for various corrective actions, such as to alert users, reallocate computational resources, and/or to adjust various data collection parameters, which may lead to an enhanced user experience, efficient device operation, and improved data integrity.

It should be understood that many variations are possible based on the disclosure herein. Although features and elements are described above in particular combinations, each feature or element is usable alone without the other features and elements or in various combinations with or without other features and elements.

What is claimed is:

1. A wearable device comprising:
a contact-based sensor configured to collect physiological data of a user;
at least one electrode positioned proximate to the contact-based sensor; and
a contact detection circuit configured to determine a contact condition of the contact-based sensor based on an electrical characteristic of a conduction path between the at least one electrode and a skin surface of the user.

2. The wearable device of claim 1, wherein the at least one electrode is positioned relative to the contact-based sensor such that contact of the at least one electrode with the skin surface correlates with contact of the contact-based sensor with the skin surface.

3. The wearable device of claim 1, wherein to determine the contact condition the contact detection circuit is configured to inject a test current through the at least one electrode and measure the electrical characteristic as a voltage used to drive the test current.

4. The wearable device of claim 3, wherein the contact detection circuit determines that the contact condition indicates that the contact-based sensor does not contact the skin surface responsive to detection of the voltage exceeding a threshold.

5. The wearable device of claim 3, wherein the contact detection circuit determines that the contact condition indicates that the contact-based sensor is in contact with the skin surface responsive to detection of the voltage as being below a threshold.

6. The wearable device of claim 1, wherein the contact condition includes a quantification of an amount of contact of the contact-based sensor with the skin surface based on a correlation to a value of the electrical characteristic.

7. The wearable device of claim 1, wherein the wearable device is configured to cause output of an indication of the contact condition, the indication including one or more of a visual notification, audio alert, or haptic feedback.

8. The wearable device of claim 1, wherein the wearable device is configured to perform data analysis on the physiological data collected by the contact-based sensor, the data analysis including weighting, filtering, or flagging one or more portions of the physiological data based on the contact condition.

9. The wearable device of claim 1, wherein the contact-based sensor is a photoplethysmography (PPG) sensor and the at least one electrode includes a first electrode positioned adjacent to a first side of the PPG sensor and a second electrode positioned adjacent to a second side of the PPG sensor.

10. A method implemented by a wearable device attachable to a skin surface of a user, the method comprising:
injecting a test current through a first electrode positioned adjacent to a contact-based sensor, the contact-based sensor configured to collect physiological data via contact with the skin surface;
determining an electrical characteristic that includes a voltage value to drive the test current to a second electrode through a conduction path that includes one or more of the first electrode, the skin surface, or the second electrode; and
determining a contact condition of the contact-based sensor based on the voltage value that indicates whether the contact-based sensor is in contact with the skin surface.

11. The method of claim 10, wherein the first electrode is positioned relative to the contact-based sensor such that contact of the first electrode with the skin surface correlates with contact of the contact-based sensor with the skin surface.

12. The method of claim 10, wherein the contact condition further includes a quantification of an amount of contact of the contact-based sensor with the skin surface based on a correlation to the voltage value.

13. The method of claim 10, further comprising causing the contact-based sensor to pause collection of the physiological data responsive to a determination that the contact-based sensor is not in contact with the skin surface.

14. The method of claim 10, further comprising causing presentation of an indication of the contact condition, the indication including one or more visual notifications, audio alerts, or haptic feedback.

15. The method of claim 14, wherein the indication further includes diagnostic information about a cause of the contact condition and user guidance to improve contact between the contact-based sensor and the skin surface.

16. The method of claim 10, wherein the contact-based sensor is a photoplethysmography (PPG) sensor, and one or more of the first electrode or the second electrode are integrated into an electrocardiogram (ECG) measurement component.

17. A contact detection system comprising:
a contact-based sensor configured to collect physiological data via contact with a skin surface;
an electrode arrangement that includes at least one proximate electrode positioned proximate to the contact-based sensor such that contact of the at least one proximate electrode with the skin surface correlates with contact of the contact-based sensor with the skin surface; and
one or more processors configured to determine a contact condition of the contact-based sensor based on an electrical characteristic of a conduction path between the at least one proximate electrode and the skin surface.

18. The contact detection system as described in claim 17, wherein the electrode arrangement includes a drive electrode configured to drive a test current and a passive electrode configured to detect the electrical characteristic resulting from the test current.

19. The contact detection system as described in claim 17, wherein the electrode arrangement includes three or more electrodes and the one or more processors are configured to identify whether each of the three or more electrodes maintain contact with the skin surface.

20. The contact detection system as described in claim 17, wherein the contact-based sensor includes one or more of a photoplethysmography (PPG) sensor, a temperature sensor, a heat flux sensor, or an optical sensor.
